(19) United States
(12) Patent Application Publication
(10) Pub. No.: US 2001/0048930 A1
(43) Pub. Date: Dec. 6, 2001

(54) Methods of immunosuppression

(76) Inventors: Jonathan Robert Lamb, Edinburgh (GB); Margaret Jane Dallman, Action (GB); Gerard Francis Hoyne, Musselburgh (GB)

Correspondence Address: FROMMER LAWRENCE & HAUG LLP, 745 Fifth Avenue, New York, NY, 10151, US

(21) Appl. No.: 09/870,902

(22) Filed: May 31, 2001

Related U.S. Application Data
(63) Continuation of application No. PCT/GB99/04233, filed on Dec. 15, 1999.

(51) Int. Cl. A61K 39/35; A61K 39/36

(52) U.S. Cl. 424/275.1; 424/93.7

(57) ABSTRACT

A method for producing a T cell having tolerance to an allergen or antigen which method comprises incubating the T cell with an antigen presenting cell (APC) in the presence of (i) a composition capable of upregulating expression of an endogenous Notch ligand in the APC and (ii) the allergen or antigen is provided.

### FIELD OF THE INVENTION

The present invention relates to methods for preparing antigen presenting cells and lymphocytes, particularly but not exclusively regulatory T cells, that can suppress the activity of lymphocytes and other cells of the immune system. It also relates to the use of compositions capable of upregulating expression of an endogenous Notch or Notch ligand in such methods. These compositions, antigen presenting cells and lymphocytes may be used in immunotherapy.

### BACKGROUND TO THE INVENTION

Immunological tolerance to self-antigens is vital to the proper functioning of the mammalian immune system. In addition to the deletion of self-reacting T cells in the thymus, active suppression mediated by regulatory T cells has recently been identified as an important mechanism for maintaining peripheral tolerance (WO98/20142). In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

It has recently been shown that it is possible to generate a class of regulatory T cells which are able to transmit antigen-specific tolerance to other T cells, a process termed infectious tolerance (WO 98/20142). The functional activity of these cells can be mimicked by over-expression of a Notch ligand protein on their cell surfaces. In particular, regulatory T cells can be generated by over-expression of a member of the Delta or Serrate family of Notch ligand proteins. Delta or Serrate expressing T cells specific to one antigenic epitope are also able to transfer tolerance to T cells recognising other epitopes on the same or related antigens, a phenomenon termed “epitope spreading”. WO98/20142 describes methods for generating regulatory T cells by either transfecting hybridoma T cells with a nucleic acid construct directing the expression of Delta or by transfecting antigen presenting cells such as dendritic cells with a nucleic acid construct directing the expression of Serrate and incubating the dendritic cells with T cells.

### SUMMARY OF THE INVENTION

The present invention identifies substances capable of upregulating expression of the endogenous genes encoding Notch or Notch ligands in antigen presenting cells (APCs) and lymphocytes. We believe that incubating APCs and lymphocytes, e.g. T cells, in the presence of these substances and a specific antigen produces APCs capable of inducing immunological tolerance in such lymphocytes or other APCs to the specific antigen. Furthermore, we believe that administration of these APCs and/or lymphocytes to a recipient individual may induce immunotolerance in that individual to the antigen. In particular we believe that immunosuppressive cytokines (such as IL-4, IL-10, IL-13, TGF-β and SLIP3 ligand) can be used to upregulate the expression of endogenous Notch or Notch ligands in APCs or lymphocytes. The present invention applies these findings to the generation of primed APCs and lymphocytes, e.g. regulatory T cells, using ex vivo methods. The resulting primed APCs and/or lymphocytes, e.g. regulatory T cells, may be readministered to the patient to treat or prevent a range of immune disorders resulting from inappropriate lymphocyte activity, such as auto-immune disease and graft rejection.

Accordingly the present invention provides a method for producing a lymphocyte or antigen presenting cell (APC) having tolerance to an allergen or antigen which method comprises incubating a lymphocyte or APC obtained from a human or animal patient with (i) a composition capable of upregulating expression of an endogenous Notch or Notch ligand in the lymphocyte and/or APC and (ii) the allergen or antigen.

According to a preferred embodiment the method comprises incubating a lymphocyte or APC obtained from a human or animal patient with an APC in presence of (i) a composition capable of upregulating expression of an endogenous Notch or Notch ligand in the lymphocyte and/or APC and (ii) the allergen or antigen.

Preferably the lymphocyte is a T cell or a B cell. Most preferably the lymphocyte is a T cell.

Accordingly the present invention further provides a method for producing ex vivo a T cell having tolerance to an allergen or antigen which method comprises incubating a T cell obtained from a human or animal patient with an antigen presenting cell (APC) in the presence of (i) a composition capable of upregulating expression of an endogenous Notch and/or Notch ligand in the APC and/or T cell and (ii) the allergen or antigen.

Preferably, the composition comprises a polypeptide selected from Noggin, Chordin, Follistatin, Xnr3, FGF and derivatives, fragments, variants and homologues thereof, and immunosuppressive cytokines, or a combination thereof. More preferably, the composition comprises at least one polypeptide selected from Noggin, Chordin, Follistatin, Xnr3, fibroblast growth factors and derivatives, fragments, variants and homologues thereof, and/or at least one immunosuppressive cytokine. Particularly preferred examples of immunosuppressive cytolines for use in the present invention are IL-4, IL-10, IL 13, TGF-β and FLT3 ligand.

The Notch ligand is preferably selected from Serrate, Delta and homologues thereof, more preferably Serrate and Delta.

The present invention also provides a second method for producing ex vivo a lymphocyte or APC having tolerance to an allergen or antigen which method comprises incubating a lymphocyte or APC obtained from a human or animal patient with a lymphocyte or APC produced by the above methods of the invention.

APCs or lymphocytes, preferably T cells, produced ex vivo by the methods of the invention may be used in suppressing an immune response in a mammal to the allergen or antigen, such as an auto-immune response or allograft rejection.

We have identified substances and combinations of substances capable of upregulating expression of an endogenous Notch or Notch ligand in an APC or lymphocyte, e.g. a T cell, and shown that such substances may be used to produce APCs and lymphocytes, such as regulatory T cells, capable of suppressing the activity of other APCs and lymphocytes, such as T cells.

Accordingly, the present invention also provides the use of a composition capable of upregulating expression of an endogenous Notch or Notch ligand in an APC and/or lymphocyte in a method of producing APCs or regulatory lymphocytes, preferably T cells, capable of suppressing the activity of other APCs and/or lymphocytes. Typically, the composition is used in vitrolex vivo rather than in vivo and the resulting APCs/lymphocytes (T cells) subsequently administered to a patient.

Thus the present invention also provides a method of treating a patient suffering from a disease characterised by inappropriate lymphocyte activity which method comprises administering to the patient a Iymphocyte produced by the methods of the invention.

It is not necessary to incubate the APC and lymphocyte simultaneously: for example, the APC can be primed first in the presence of the antigen and substance capable of upregulating Notch or Notch ligand expression. When such a primed APC is contacted with a lymphocyte, either in vitro, ex vivo or in vivo, tolerance to the antigen is induced in the lymphocyte.

Accordingly, the present invention further provides a method for producing an antigen presenting cell (APC) capable of inducing in a lymphocyte tolerance to an allergen or antigen which method comprises contacting an APC with (i) a composition capable of upregulating expression of an endogenous Notch or Notch ligand in the APC or lymphocyte and (ii) the allergen or antigen. Such APCs may be administered to a patient in a method of immunotherapy. Preferably the method is carried out ex vivo preferably using APCs or lymphocytes obtained from a human or animal patient suffering from a immune disorder or the recipient of a tissue graft/organ transfer.

Also provided is a method for producing ex vivo a lymphocyte having tolerance to an allergen or antigen which method comprises incubating an APC, produced as described above, with the lymphocyte.

In one preferred embodiment step (i) comprises introducing a nucleic acid sequence into the lymphocyte or APC, which is capable of upregulating expression of an endogenous Notch or Notch ligand, preferably by expression of a polypeptide which is capable of upregulating expression of an endogenous Notch or Notch ligand.

Preferably the nucleic acid sequence encodes a polypeptide selected from Noggin, Chordin, Follistatin, Xnr3, fibroblast growth factors and derivatives, fragments, variants and homologues thereof, and/or at least one immunosuppressive cytokine. Particularly preferred examples of immunosuppressive cytokines for use in the present invention are IL-4, IL-10, IL-1 3, TGF-β and FLT3 ligand.

Alternatively, the nucleic acid sequence is an antisense construct derived from a sense nucleotide sequence encoding a polypeptide selected from a polypeptide capable of downregulating Notch or Notch ligand expression, such as Toll-like receptors, a cytokine such as IL12, IFN-γ, TNF-α or a growth factor such as a BMP or a BMP receptor and activins.

In another preferred embodiment the composition is a chemical compound such as a polypeptide which is exposed/incubated with the lymphocyte or APC. The agent should be one which is capable of modulating Notch-Notch ligand interactions. In this embodiment the polypeptide is preferably selected from from Noggin, Chordin, Follistatin, Xnr3, fibroblast growth factors and derivatives, fragments, variants and homologues thereof, and/or at least one immunosuppressive cytokine. Particularly preferred examples of immunosuppressive cytokines for use in the present invention are IL-4, IL-10, IL-13, TGF-β and FLT3 ligand Preferably when the composition is a receptor or a nucleic acid sequence encoding a receptor, the receptor is activated. Thus, when the agent is a nucleic acid sequence, the receptor is constitutively active when expressed.

As used herein, the terms protein and polypeptide may be assumed to be synonymous, protein merely being used in a general sense to indicate a relatively longer amino acid sequence than that present in a polypeptide.

The term “derivative” as used herein, in relation to proteins or polypeptides of the present invention includes any substitution of, variation of, modification of, replacement of, deletion of or addition of one (or more) amino acid residues from or to the sequence providing that the resultant protein or polypeptide possesses the capability of modulating Notch-Notch ligand interactions.

The term “variant” as used herein, in relation to proteins or polypeptides of the present invention includes any substitution of, variation of, modification of, replacement of, deletion of or addition of one (or more) amino acid residues from or to the sequence providing that the resultant protein or polypeptide possesses the capability of modulating Notch-Notch ligand interactions.

The term “analog” are used herein, in relation to the proteins or polypeptides of the present invention includes any peptidomimetic, that is, a chemical compound that possesses the capability of modulating Notch-Notch ligand interactions in a similar manner to the parent protein or polypeptide. These include compounds that may antagonise the expression or activity of a Notch-protein or Notch-ligand.

An agent may be considered to modulate Notch-Notch ligand interactions if it is capable of promoting the interaction of Notch with its ligands, preferably to an extent sufficient to provide therapeutic efficacy.

In a preferred embodiment the agent modulates Notch-Notch ligand interactions by being capable of upregulating expression of Notch or Notch ligand.

The expression “Notch-Notch ligand” as used herein means the interaction between a Notch family member and a ligand capable of binding to one or more such member. Thus by the expression “upregulating interaction of Notch or a Notch-ligand” we mean promoting the interaction of Notch in a lymphocyte or APC with a Notch ligand or promoting the interaction of a Notch ligand in a lymphocyte or APC with Notch. Preferably the lymphocyte is a T cell.

The term therapy are used herein should be taken to encompass diagnostic and prophylatic applications.

The present invention further provides a pharmaceutical composition comprising a primed APC and/or lympocyte of the invention together with a pharmaceutically acceptable carrier or diluent.

### DETAILED DESCRIPTION OF THE INVENTION

Various preferred features and embodiments of the present invention will now be described by way of non-limiting example.

A. Notch and Notch Ligands

An endogenous Notch ligand in the context of the present invention is a polypeptide encoded by the genome of a mammalian cell that is capable of being expressed by the mammalian cell. In particular the mammalian cell may be a haemapoietic cell such as a T cell or an antigen presenting cell. The endogenous Notch ligand is typically is capable of binding to a Notch receptor polypeptide present in the membrane of a variety of mammalian cell types, for example haemapoietic stem cells. At least four Notch receptors (Notch-1, Notch-2, Notch-3 and Notch-4) have been identified to date in human cells.

Particular examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive. For example, human Jagged-2 has 40.6% identity and 58.7% similarity to Serrate.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and between 3 to 8 EGF-like repeats on the extracellular surface. It is therefore preferred that homologues of Notch ligands also comprise a DSL domain at the N-terminus and between 3 to 8 EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

B. Substances Capable of Upregulating Endogenous Notch or Notch ligand Expression

Substances that may be used to upregulate Notch ligand expression include polypeptides that bind to and reduce or neutralise the activity of bone morphogenetic proteins (BMPs). Binding of extracellular BMPs (Wilson and Hemmati-Brivanlou, 1997, Hemmati-Brivanlou and Melton, 1997) to their receptors leads to down-regulated Delta transcription due to the inhibition of the expression of transcription factors of the achaete/scute complex. This complex is believed to be directly involved in the regulation of Delta expression. Thus, any substance that inhibits BMP expression and/or inhibits the binding of BMPs to their receptors may be capable of producing an increase in the expression of Notch ligands such as Delta and/or Serrate. Particular examples of such inhibitors include Noggin (Valenzuela et al., 1995), Chordin (Sasai et al., 1994), Follistatin (Iemura et al., 1998), Xnr3, and derivatives and variants thereof Noggin and Chordin bind to BMPs thereby preventing activation of their signalling cascade which leads to decreased Delta transcription. Consequently, increasing Noggin and Chordin levels may lead to increase Notch ligand, in particular Delta, expression.

Furthermore, any substance that upregulates expression of transcription factors of the achaete/scute complex may also upregulate Notch ligand expression.

Other suitable substances that may be used to upregulate Notch ligand expression include transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao et al., 1995). Most preferably, the FGF is a member of the FGF family which acts by stimulating the upregulation of expression of a Serrate polypeptide on APCs. The inventors have shown that members of the FGF family can upregulate Serrate-1 gene expression in APCs.

Immunosuppressive cytokines may also be used to upregulate Notch ligand expression. Examples include members of the TGF-β family such as TGF-β-1 and TGF-β-2, and interleukins such as IL-4, IL-10 and IL-13, and FLT3 ligand.

The inventors have shown that: members of the TGF-β family can upregulate Notch, particularly Notch 1, expression in CD4+ T cells;

IL-10 can upregulate serrate, particularly Serrate 1, gene expression in dentritic cells;

IL-10 can upregulate Notch, Delta and Serrate, particularly Notch 2, Notch 4, Delta 1 and Serrate 1, gene expression in naive B cells; and

IL-10 can upregulate Serrate, particularly Serrate 1, gene expression in mature DCs.

Generally the inventors have shown that selected cytokines affect different receptors/ligands in different ways, and that this also varies between cell type and tissue.

The substance capable of upregulating expression of Notch or a Notch ligand may be selected from polypeptides and fragments thereof, linear peptides, cyclic peptides, synthetic and natural compounds including low molecular weight organic or inorganic compounds. The substances capable of upregulating expression of a Notch ligand may be derived from a biological material such as a component of extracellular matrix. Suitable extracellular matrix components are derived from immunologically privileged sites such as the eye. For example aqueous humour or components thereof may be used.

Polypeptide substances such as Noggin, FGFs and TGF-β may be purified from mammalian cells, obtained by recombinant expression in suitable host cells or obtained commercially. Alternatively, nucleic acid constructs encoding the polypeptides may be introduced into APCs and/or lymphocytes (T cells) by transfection using standard techniques or viral infection/transduction. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the APC or lymphocte (T cell).

It is particularly preferred to use combinations of substances, for example a combination of at least two substances. In a preferred embodiment, an immunosuppressive cytokine is used in combination with another substance capable of upregulating Notch ligand expression. Other examples of preferred combinations include at least one substance capable of upregulating Serrate expression (such as FGF), preferably in an APC, together with at least one substance capable of upregulating Delta expression (such as Noggin or Chordin), preferably in a T cell. Alternatively, a preferred combination comprises at least one substance which acts via inhibition of binding of BMPs to their receptors together with at least one substance which has a different mode of action.

Preferably, the composition, preferably a nucleic acid sequence, for use in the present invention is capable of upregulating Serrate and Delta, preferably Serrate 1 and Serrate 2 as well as Delta 1 and Delta 3 expression in APCs such as dendritic cells.

Preferably, the substance for use in the present invention is capable of upregulating Serrate expression in APCs such as dendritic cells. In particular, the substance may be capable of upregulating Serrate expression but not Delta expression in APCs. Alternatively, the substance for use in the present invention is capable of upregulating Delta expression in T cells such as CD4⁺ helper T cells or other cells of the immune system that express Delta (for example in response to stimulation of cell surface receptors). In particular, the substance may be capable of upregulating Delta expression but not Serrate expression in T cells. In a particularly preferred embodiment, the substance is capable of upregulating Notch ligand expression in both T cells and APC, for example Serrate expression in APCs and Delta expression in T cells.

Suitable substances for use according to the present invention may be conveniently identified using a simple screening procedure. In one such assay procedure, lymphocytes, such as T cells, or APCs in culture may be contacted with a candidate substance and the effect on expression of an endogenous Notch ligand, such as Delta or Serrate, determined, for example by (i) measuring transcription initiated from the gene encoding the Notch ligand as described in the Examples or by quantitative-reverse transcriptase-polymerase chain reaction (RT-PCR); (ii) detecting Notch ligand protein by techniques such as Western blotting of cell extracts, immunohistochemistry or flow cytometry; and/or (iii) functional assays such as cell adhesion assays.

The present invention also relates to modification of Notch-protein expression or presentation on the cell membrane or signalling pathways. Agents that enhance the presentation of a fully functional Notch-protein on the lymphocyte or APC surface include matrix metalloproteinases such as the product of the Kuzbanian gene of Drosophila (Dkuz et al (1997)) and other ADAMALYSIN gene family members.

In more detail, whether a substance can be used for modulating Notch-Notch ligand expression may be determined using suitable screening assays.

Screening assays for the detection of increased Notch, Notch ligand expression and/or processing include:

Notch-Notch ligand expression may be assessed following exposure of isolated cells to test compounds in culture using for example:

(a) at the protein level by specific antibody staining using immunohistochemistry or flow cytometry.

(b) at the RNA level by quantitative—reverse transcriptase-polymerase chain reaction (RT-PCR). RT-PCR may be performed using a control plasmid with in-built standards for measuring endogenous gene expression with primers specific for Notch 1 and Notch 2, Serrate 1 and Serrate 2, Delta 1 and Delta 2 and Delta 3. This construct may be modified as new ligand members are identified.

(c) at the functional level in cell adhesion assays.

Increased Notch ligand or Notch expression should lead to increased adhesion between cells expressing Notch and its ligands. Test cells will be exposed to a particular treatment in culture and radiolabelled or flourescein labelled target cells (transfected with Notch/Notch ligand protein) will be overlayed. Cell mixtures will be incubated at 37° C. for 2 hours. Nonadherent cells will be washed away and the level of adherence measured by the level of radioactivity/imrnmunofluorescence at the plate surface.

Using such methods it is possible to detect compounds or Notch-ligands that affect the expression or processing of a Notch-protein or Notch-ligand. The invention also relates to compounds, or Notch-ligands detectable by these assays methods, and also to their use in the methods of the present invention.

These procedures may also be used to identify particularly effective combinations of substances for use according to the present invention.

C. Antigen Presenting Cells and Lymphocytes

Antigen-presenting cells (APCs) for use in the present invention may be “professional” antigen presenting cells or may be another cell that may be induced to present antigen to T cells. Alternatively a APC precursor may be used which differentiates or is activated under the conditions of culture to produce an APC. The APC may be isolated from a patient, or recipient of the immunotherapy or from a donor individual or another individual. Preferably the APC or precursor is of human origin. If the APC or precursor APC is from a different individual to the T cells, the donor APC may also serve as the source of antigen.

APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, activated to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation. Such proteins include MHC molecules (Class I or Class II), CD80, CD86, or CD40. Most preferably DCs or DC-precursors are included as a source of APCs.

The APC or precursor APC may be provided by a cell proliferating in culture such as an established cell line or a primary cell culture. Examples include hybridoma cell lines, L-cells and human fibroblasts such as MRC-5. Cell lines may conveniently be used in the screening procedures described above.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al., 1992), or from bone marrow, non-adherent CD34⁺ cells can be treated with GM-CSF and TNF-α (Caux et al., 1992). DCs can also be routinely prepared from the peripheral blood of human volunteers, similarly to the method of Sallusto and Lanzavecchia (1994) using purified peripheral blood mononucleocytes (PBMCs) and treating 2 hour adherent cells with GM-CSF and IL-4. If required, these may be depleted of CD19⁺ B cells and CD3⁺, CD2⁺T cells using magnetic beads (see Coffin et al., 1998). Culture conditions may include other cytokines such as GM-CSF or IL-4 for the maintenance and, or activity of the dendritic cells or other antigen presenting cells.

Where lymphocytes are to be used they are preferably T cells or B cells. T cells are most preferred.

Where T cells or B cells are to be used in ex vivo methods of inducing immunotolerance, the T cells or B cells for use in the invention are typically isolated from an individual suffering from a disease of the immune system or a recipient for a transplant operation or from a related or unrelated donor individual. T cells or B cells may be obtained from blood or another source (such as lymph nodes, spleen, or bone marrow) and may be enriched or purified by standard procedures. The T cells or B cells may be used in combination with other immune cells, obtained from the same or a different individual. Alternatively whole blood may be used or leukocyte enriched blood or purified white blood cells as a source of T cells, B cells and other cell types. It is particularly preferred to use helper T cells (CD4⁺). Alternatively other T cells such as CD8⁺ cells may be used.

Where T cells or B cells are to be used in in vitro screening procedures, it may be convenient to use cell lines, such as T cell hybridomas.

Thus, it will be understood that the term “antigen presenting cell or the like” as used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

E. Antigens and Allergens

An antigen may be any substance that can be recognised generally as foreign, by the immune system, and is generally recognised by an antigen receptor. Preferably the antigen of the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (THI) is characterised by high IL-2 and IFNγ but low IL-4 production, whereas in humoral immunity (TH2) the pattern is low IL-2 and IFNγ but high IL-4, IL-5, IL-10. Since the secretory pattern is modulated at the level of the secondary lymphoid organ or cells, then pharmacological manipulation of the specific TH cytokine pattern can influence the type and extent of the immune response generated.

The TH1-TH2 balance refers to the interconversion of the two different forms of helper T cells. The two forms have large scale and opposing effects on the immune system. If an immune response favours THI cells, then these cells will drive a cellular response, whereas TH2 cells will drive an antibody-dominated response. The type of antibodies responsible for some allergic reactions is induced by TH2 cells.

The present invention has uses in relation to both responses.

The antigen or allergen may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens. Where primed APCs/ T cells of the present invention are to be used in tissue transplantation procedures, antigens will be obtained from the tissue donor.

The antigen or allergen moiety may be, for example, a synthetic MHC-peptide complex i.e. a fragment of the MHC molecule bearing the antigen groove bearing an element of the antigen. Such complexes have been described in Altman et al., 1996.

F. Preparation of Primed APCs and Lymphocytes

1. Preparation of Primed APCs ex vivo in the Absence of Lymphocytes

APCs as described above are cultured in a suitable culture medium such as DMEM or other defined media, optionally in the presence of fetal calf serum. Cytokines, if present, are typically added at up to 1000 U/ml. Optimum concentrations may be determined by titration. One or more substances capable of upregulating Notch or Notch ligand expression are then typically added to the culture medium together with the antigen of interest. The antigen may be added before, after or at substantially the same time as the substance(s). Cells are typically incubated with the substance(s) and antigen for at least one hour, preferably at least 3 hours, at 37° C. If required, a small aliquot of cells may be tested for upregulation of Notch or Notch ligand expression as described above. Alternatively, cell activity may be measured by the inhibition of T cell proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction.

The resulting APCs that express increased levels of a Notch ligand and are presenting antigen on their cell surface complexed with MHC are now ready for use. For example, they may be prepared for administration to a patient or incubated with T cells in vitro (ex vivo) to induce immunotolerance in the T cells as described in WO98/20142.

2. Preparation of Regulatory T cells (and B cells) ex vivo

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s) capable of upregulating Notch or Notch ligand expression and/or cytokine to that used with the APC, then the T cell will not be brought into contact with the different substance(s) used to upregulate Notch or Notch ligand expression in the APC. Alternatively, the T cell may be incubated with the substance(s)/cytokine first to induce Notch or Notch ligand expression, washed, resuspended and then incubated with the primed APC in the absence of both the substance(s) used to upregulate APC Notch ligand expression and the substance(s) used to upregulate Notch or Notch ligand expression in the T cell. Once primed APCs have been prepared, it is not always necessary to administer any substances to the T cell since the primed APC is itself capable of inducing immunotolerance leading to increased Notch or Notch ligand expression in the T cell, presumably via Notch/Notch ligand interactions between the primed APC and T cell.

Incubations will typically be for at least 1 hour, preferably at least 3 or 6 hours, in suitable culture medium at 37° C. The progress of induction of Notch or Notch ligand expression may be determined for a small aliquot of cells using the methods described above. T cells transfected with a nucleic acid construct directing the expression of, for example Delta, may be used as a control. Induction of immunotolerance may be determined by subsequently challenging T cells with antigen and measuring L-2 production compared with control cells not exposed to APCS.

Primed T cells or B cells may also be used to induce immunotolerance in other T cells or B cells in the absence of APCs using similar culture techniques and incubation times. Generally, the addition of substances capable of upregulating Notch or Notch ligand expression is not required at this stage but they may be added if desired, together with immunosuppressive cytokines.

G. Transgenic Animals

The present invention also relates to cell lines or transgenic animals which are capable of expressing or overexpressing Notch, a Notch ligand or at least one agent useful in the present invention. Preferably the cell line or animal expresses or overexpresses Notch, Delta or Serrate.

The present invention additionally relates to cell lines or transgenic animals which are capable of expressing or overexpressing at least one polypeptide which is capable of promoting Notch-Notch ligand interactions. Such agents have been described above and for the avoidance of doubt are specifically incorporated herein by reference.

The present invention further relates to cell lines or transgenic animals which are capable of expressing or overexpressing at least one polypeptide which is capable of enhancing Notch-Notch ligand interactions. Agents that enhance the presentation of a fully functional Notch-protein on the lymphocyte or APC surface include matrix metalloproteinases such as the product of the Kuzbanian gene of Drosophila (Dkuz et al., (1997) and other ADAMALYSIN gene family members. Suitable agents that influence expression of Notch-ligands include agents that affect the expression of Delta and/or Serrate genes. For instance, for Delta expression, any agent that inhibits the binding of BMPs to their receptors is capable of producing an increase in the expression of Delta and/or Serrate. Such agents include Noggin, Chordin, Follistatin, FGFs, Fringe and derivatives and variants thereof.

The transgenic animal is typically a vertebrate, more preferably a rodent, such as a rat or a mouse, but also includes other mammals such as human, goat, pig or cow etc.

Such transgenic animals are useful as animal models of disease and in screening assays for new useful compounds. By specifically expressing one or more polypeptides, as defined above, the effect of such polypeptides on the development of disease can be studied. Furthermore, therapies including gene therapy and various drugs can be tested on transgenic animals. Methods for the production of transgenic animals are known in the art. For example, there are several possible routes for the introduction of genes into embryos. These include (i) direct transfection or retroviral infection of embryonic stem cells followed by introduction of these cells into an embryo at the blastocyst stage of development; (ii) retroviral infection of early embryos; and (iii) direct microinjection of DNA into zygotes or early embryo cells.

The present invention also includes stable cell lines for use as disease models for testing or treatment. A stable cell line will contain a recombinant gene or genes, also known herein as a transgene.

A cell line containing a transgene, as described herein, is made by introducing the transgene into a selected cell line according to one of several procedures known in the art for introducing a foreign gene into a cell.

The sequences encoding the inhibitors and enhancers of Notch-Notch ligand interactions as well as Notch or a Notch ligand itself are operably linked to control sequences, including promoters/enhancers and other expression regulation signals.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the lung epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

H. Therapeutic Uses

We have shown that APCs and lymphocytes expressing Notch and Notch ligands are capable of efficiently transferring infectious tolerance to the chosen antigen or antigens when transferred into the patient for the treatment of a disease characterised by inappropriate lymphocyte activity, such as Th1 or Th2 cell activity. The APCs and/or lymphocytes may thus be used to treat an ongoing immune response (such as an allergic condition or an autoimmune disease) or may be used to generate tolerance in an immunologically lymphocytes cells of the present invention may be used in therapeutic methods for both treating and preventing diseases characterised by inappropriate lymphocyte activity in animals and humans. The APCs and/or lymphocytes may be used to confer tolerance to a single antigen or to multiple antigens.

Typically, APCs and/or lymphocytes are obtained from the patient or donor and primed as described above before being returned to the patient (ex vivo therapy).

Particular conditions that may be treated or prevented include multiple sclerosis, rheumatoid arthritis, diabetes, allergies, asthma, and graft rejection. The present invention may also be used in organ transplantation or bone marrow transplantation.

I. Administration

Primed APCs/lymphocytes of the present invention for use in immunotherapy are typically formulated for administration to patients with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, intra-peritoneal, injection, intranasal inhalation, lung inhalation, intradernal, intra-articular, intrathecal, or via the alimentary tract (for example, via the Peyers patches).

Cells and pharmaceutical comprising cells of the invention are typically administered to the patient by intramuscular, intraperitoneal or intravenous injection, or by direct injection into the lymph nodes of the patient, preferably by direct injection into the lymph nodes. Typically from 10⁴ to 10⁸ treated cells, preferably from 10⁵ to 10⁷ cells, more preferably about 10⁶ cells are administered to the patient.

The routes of administration and dosages described are intended only as a guide since a skilled practitioner will be able to determine readily the optimum route of administration and dosage for any particular patient depending on, for example, the age, weight and condition of the patient.

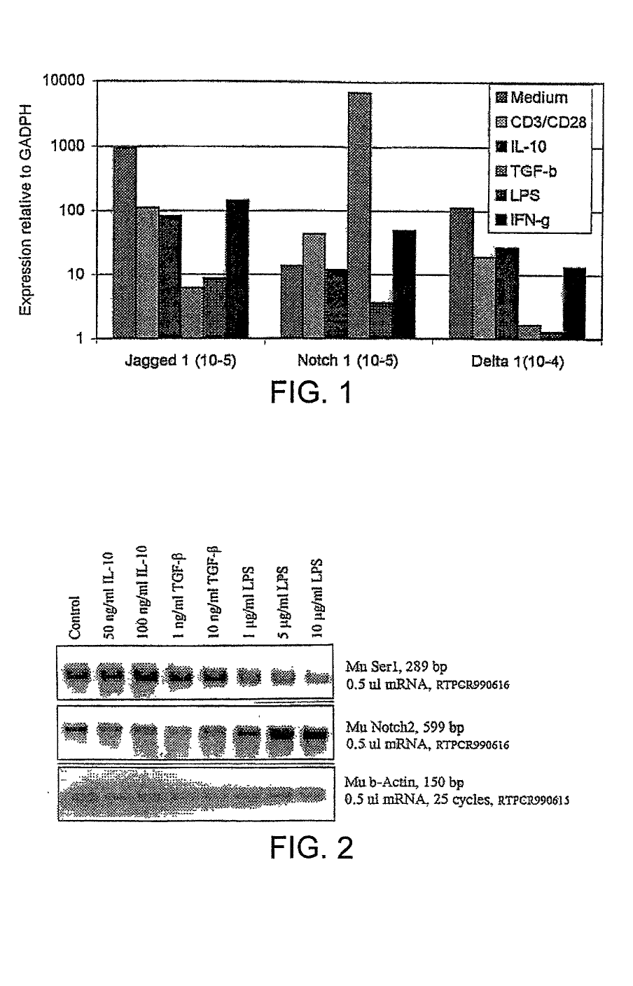

The present invention will now be described by way of examples which are intended to be illustrative only and non-limiting, and by reference to the accompanying Figures in which FIGS. 1, 2, 3 and 4 show the results of Examples 8, 9, 10 and 11 respectively.

### EXAMPLES

Materials and Methods

Construction of Retroviruses expressing Delta or Serrate

A cDNA encoding human Serrate-1 is inserted as a BamHI-EcoRI fragment into the retroviral vector pBABEneo (Morgenstern and Land, 1990) using standard techniques, such that the cDNA is expressed from the retroviral promoter element. Transducing vector particles can be produced by transfection of this construct into a suitable amphotropic packaging cell line such as PA317 (ATCC catalogue number CRL-9078), FLYA13 or FLYRD114 (Cosset et al., 1995). Alternatively a permanent cell line containing the vector genome and gag-pol gene is constructed by transfection of Serrate-pBABE vector into PA327 cells. Production of replication—deficient pseudotyped vector particles is initiated by transient transfection with a plasmid encoding the G-protein from Vesicular Stomatitis Virus (VSV) under the control of the hCMV major immediate early promoter-enhancer.

A retroviral vector expressing mouse Delta-1 is prepared in a similar fashion.

Preparation of Dendritic Cells from Mouse Spleen

Single cell suspensions are prepared from the spleens of mice. Rapidly adhering cells are isolated by culture for 2-3 h at 37° C. in plastic tissue culture flasks. Non-adherent cells are removed by extensive washing and 50 ng/ml mouse GM-CSF in culture medium is added for 24 h. Culture medium is RPMI 1640 with 2% FCS, 50 mM 2-mercaptoethanol, and optionally 0.02 mg/ml Penicillin and Streptomycin.

Preparation of T cells from Mouse

T cells are purified from blood or lymph nodes by positive selection on magnetic antibody-coated beads specific for particular cell types (MACS columns) using methods provided by the manufacturer (Miltenyi Biotech) as follows.

Lymph nodes are removed and single cell suspensions prepared in tissue culture medium (10⁸ cells in 0.4 ml RPMI 1640 with 10% FCS). Cells are incubated at 4° C. for 15 min, passed over the MACS separation column, washed and collected. CD4-positive cells are enriched by negative selection on anti-CD8 antibody-coated magnetic beads.

Determination of Serrate and Delta expression.

After various incubation times, cells (1.5×10⁶) are harvested, pelleted and frozen. RNA is prepared from cell pellets by homogenisation in guanidium thiocyanate solution followed by CsCl density centrifigation. 1 μg RNA is converted into cDNA using an oligo dT primer. Of the resultant cDNA, 1/20th was used in PCR (40 cycles) using primers specific for the human delta homologue or the human serrate homologue.

RT-PCR is performed using an Access RT-PCR kit (Promega). 50 ng RNA is used in each reaction together with Serrate-1 gene specific oligonucleotide primers (50 pmol) under conditions according to the manufacturer's instructions (Tm for the Serrate oligonucleotides is 58° C.).

The sequence of the “forward” Serrate-1 primer is:

5′-GGCTGGGAAGGAACAACCTG-3′

The Serrate- “reverse” primer is: 5′-GGTAGCCATTGATCTCATCCAC-3′

Primers specific for Delta are:

5′-GATTCTCCTGATGACCTCGC-3′

5′-GTGTTCGTCACACACGAAGC-3′

PCR samples were analysed by gel electrophoresis.

Determination of Notch, Serrate and Delta Expression

In the Examples 8-11 the following primers were used as indicated:

Murine Notch1 Accession No. Z 11886

Forward primer (FP) 5′-TGTTAATGAGTGCATCTCCAACCC-3′

Reverse primer (RP) 5′-CATTCGTAGCCATCAATCTTGTCC-3′

Murine Notch2 Accession No. D32210

FP 5′-CAGAGGAATAGCAAGACGTGCAAG-3′

RP 5′-GATGAAGAACAGGATGATGACAACAG-3′

Murine Notch4 Accession No. U43691

FP 5′-CTACTGCCACAAGTAGCTGG-3′

RP 5′-CTCGGAGATAGCGTGACTGG-3′

Murine Serrate1 (Jagged 1) Shimizu et al 1999

FP 5′-GGGGGTCACTGTCAGAATGA-3′

RP 5′-AGATATACCGCACCCCTTCAG-3′

Murine Serrate2 (Jagged 2)

FP 5′-ATCTGCGAGGACCTGGTGGAT-3′

RP 5′-TATACCAGAGGGTGCGACA-3′

Murine Delta1 Accession No. X80903

FP 5′-GACTCTCCCGATGACCTC-3′

RP 5′-GATGCACTCATCGCAGTAG-3′

### Example 1

Assays to Determine to Identify Substances that Upregulate Notch Ligand Expression.

Dendritic cells (DCs) are the primary antigen presenting cell in the immune system and are critical for stimulating T cell responses. DCs are obtained from the spleen of mice and transferred to flasks containing tissue culture medium (RPMI 1640 with 10% fetal calf serum added). Cytokines (eg IL-4 and GM-CSF) are added as appropriate.

Cells are then transferred into 12-well tissue culture trays. To each well is added a different candidate upregulator of Notch ligand expression. Delta and Serrate expression is monitored at various time points by removing an aliquot of cells and determining induction of Delta and Serrate expression by PCR.

Similar procedures are also carried out using a T cell hybridoma cell line and T cells obtained from mice as described in the materials and methods section.

### Example 2

Preparation of Primed Dendritic Cells

DCs are obtained from the spleen of mice as in Example 1 and divided into two cultures. The first culture is transfected with a retrovirus allowing expression of the fill length Serrate-1 protein to serve as a positive control. The first culture is then pulsed with the HDM peptide p110-131 for 3 hours at 37° C. The second culture is split up into several tissue culture plate wells and to each well is added a different upregulator of Notch ligand expression identified in Example 1. These wells are then also pulsed with the HDM peptide p110-131 for 3 hours at 37° C.

The DCs are then washed and used to immunise naive mice subcutaneously using 10⁵ cells/mouse. After 7 days the draining LNCs are recovered and restimulated in culture with peptide at 4×10⁵ cells/well. Since the mice were only immunised with peptide-pulsed DCs this gives us a measure of the ability of these cells to prime an immune response.

### Example 3

Upregulation of Serrate expression in antigen presenting cells prevents T cell responses.

An influenza-reactive human T cell clone HA1.7 is mixed with peptide HA306-318 (1.0 μg/ml) in the presence of L cells expressing HLA-DRB1*0101(as antigen presenting cells), using 2×10⁴ of each cell type. The L cells have been preincubated with one or more substances identified as being capable of upregulating Serrate expression in APCs for 6 hours. The proliferative response is measured after 72 hours following addition of tritiated thymidine for the last 8 hours of culture.

### Example 4

Serrate Upregulated Antigen Presenting Cells Induce Regulatory T Cells that can Block the Response of Normal T Cells.

An influenza-reactive human T cell clone HA1.7 is mixed with peptide HA306-318 and L cells (expressing DRB1*0101 as antigen presenting cells) in the presence of 2% IL-2. The L cells have been preincubated with one or more substances identified as being capable of upregulating Serrate expression in APCs for 6 hours. After 7 days in culture, the HA1.7 cells were harvested, washed and irradiated before being mixed with fresh HA1.7 (using 2×10⁴ each population). Cells are cultured for a further 2 days before being stimulated with peptide (1.0 μg/ml)+normal APCs (DRB1*0101 PBMCs). The proliferative response is measured after 72 hours following addition of tritiated thymidine for the last 8 hours of culture.

The results show the ability of cells tolerised by upregulation of Serrate to pass on their tolerance to a naive cell population (infectious/bystander tolerance).

### Example 5

Preparation of Regulatory T cells ex vivo using primed APCs

Primed dendritic cells are produced using the same method as in Example 2. These cells are then washed, pelleted and resuspended in fresh culture medium. T cells obtained from the mouse host are then incubated with the primed dendritic cells for up to 6 hours. Aliquots of cells are taken at regular intervals and Delta and Serrate expression measured. Helper T cells are separated from the other cells using magnetic beads specific for CD4 prior to pelleting and RNA extraction.

Induction of immunotolerance in the T cells is also measured in a functional assay. HDM peptide p110-131 added to the cell culture and the cells cultured for 24 hours. Supernatant fluids are then collected and assayed for IL-2 (a major T cell growth factor) content.

### Example 6

Delta-upregulated Primed T Cells are able to Inhibit the Development of Immunty to Peptide 110-131 Antigen in Animals.

1×10⁷ primed regulatory T cells generated by the method described in Example 4 are injected into C57 BL mice i.p. The mice are also immunised with 50 μg Der p1 emulsified in Complete Freunds Adjuvant (CFA) sub-cutaneously. After 7 days the draining lymph node cells are collected and cultured at 4×10⁵ cells/well with Der p1 (10 μg /ml) or peptide 110-131 of Der p1 (10 μg /ml). Cultures are incubated at 37° C. for 72 hours and tritiated thymidine added for the final 8 hours of culture.

The results show that the primed regulatory T cells inhibit the development of an immune response to the Der p1 antigen in the immunised mouse.

### Example 7

Treatment of Patients Undergoing Bone Marrow Transplantation

Donor individuals for the bone marrow transplantation procedure are selected from an appropriate category (live related; MHC-matched un-related or unmatched); DCs are isolated from the donor by a suitable method (eg as described in U.S. Pat. No. 5,789,148) between 14 days prior to, and 3 days after, transplantation. DCs are maintained in culture in tissue culture medium eg RPMI-1640 supplemented with up to 10% autologous or ABO human serum). Inducers of Notch-ligand expression are added for the appropriate time (between 3 h and 2 days). Cytokines are also added as required (eg IL-4 and GM-CSF).

DCs may be similarly prepared from the transplant recipient if required.

Lymphocytes are obtained by an appropriate method (e.g. according to the procedures described in US-A-4663058) from the donor and/or recipient. T cells may be enriched by standard methods including antibody-mediated separation. Cells are cultured in RPMI-1640 with serum (autologous or ABO human serum) together with DCs.

T cells and DCs are then transferred to the transplant recipient by infusion at a suitable time, between 14 days before and 3 days after transplantation.

Other modifications of the present invention will be apparent to those skilled in the present art.

### Example 8

Modulation of the Expression of Notch Receptors and Ligands on Activated Murine CD4+ T Cells in Response to Inflammatory and Immunosuppressive Stimuli

CD4+ T cells were separated by MACS column from the spleens of naive BALBiC mice. Cells (2×10⁶/ml) were cultured for 48 hours in tissue culture medium (RPMI 1640) supplemented with penicillin/streptomycin, L-glutamine and 5% foetal calf serum) at 37° C. and activated by 5μg/ml plated anti-CD3 and 5μg/ml soluble anti-CD28 antibodies alone or together with interleukin 10, IL-10 (80 ng/ml), lipopolysaccharide, LPS (10 μg/ml), transforming growth factor β, TGF-β (10 ng/ml) or interferon γ, IFN-γ (10 ng/ml). Cells were collected and centrifuged at 1500 rpm and MRNA was isolated using Oligotex following the manufacturer's instructions, transcribed into cDNA and analysed by real time PCR (TaqMan, ABI) following the manufacturer's instructions.

Results The results are illustrated in FIG. 1 reveal the following changes in transcript levels for activated CD4+ T cells:

| | | | | | |
|---|---|---|---|---|---|
| Serrate1 | IL-10 | no change | Delta1 | IL-10 | no change |
| | TGF-β | decreased | | TGF-β | decreased |
| | LPS | decreased | | LPS | decreased |
| | IFN-γ | no change | | IFN-γ | no change |
| Notch 1 | IL-10 | decreased | | | |
| | TGF-β | decreased | | | |
| | LPS | decreased | | | |
| | IFN-γ | no change | | | |

### Example 9

Modulation of the Expression of Notch Receptors and Ligands on Naive Murine Dendritic Cells (DCs) in Response to Inflammatory and Immunosuppressive Stimuli

CD11c+ DCs were MACS separated from the spleens of naive BALB/C mice and incubated at 37° C. for 24 hours in medium alone or together with IL-10 (50 and 10 ng/ml), lipopolysaccharide (LPS; 1, 5 and 10 μg/ml) or TGF-β (1 and 10 ng/ml). Cells were collected and centrifuged at 1500 rpm and mRNA was isolated using Oligotex following the manufacturer's instructions. RT-PCR was performed using an Access RT-PCR kit (Promega). One μg of total RNA was used in each reaction together with specific oligonucleotide primers (50 pmol) for the following genes as indicated under conditions according to the manufacturer's instructions. PCR was performed using a Hybaid machine, dynazyme II polyrnerase, 1.5 mM Mg, 28-35 cycles at an annealing temperature between 56-63° C.

Results: The results are illustrated in FIG. 2 and reveal the following changes in transcript levels for naive splenic CD11c+ DCs

| | | | | | |
|---|---|---|---|---|---|
| Serrate1 | IL-10 | increased | Notch2 | IL-10 | no change |
| | TGF-β | no change | | TGF-β | no change |
| | LPS | decreased | | LPS | increased |

### Example 10

Modulation of the Expression of Notch Receptors and Ligands on naive B Cells in Response to Inflammatory and Immunosuppressive Stimuli

B cells were MACS separated from the spleens of naive BALB/C mice and incubated at 37° C. for 24 hours in medium alone or together with IL-10 (80ng/ml) or lipopolysaccharide (LPS; 5 and 10 μg/ml). Cells were collected and centrifuged at 1500 rpm and mRNA was isolated using Oligotex following the manufacturer's instructions. RT-PCT was performed using an Access RT-PCR kit (Promega). One μg of total RNA was used in each reaction together with specific oligonucleotide primers (50 pmol) for the following genes as indicated under conditions according to the manufacturer's instructions. PCR was performed using a Hybaid machine, dynazyme II polymerase, 1.5 mM Mg, 28-35 cycles at an annealing temperature between 56-63° C.

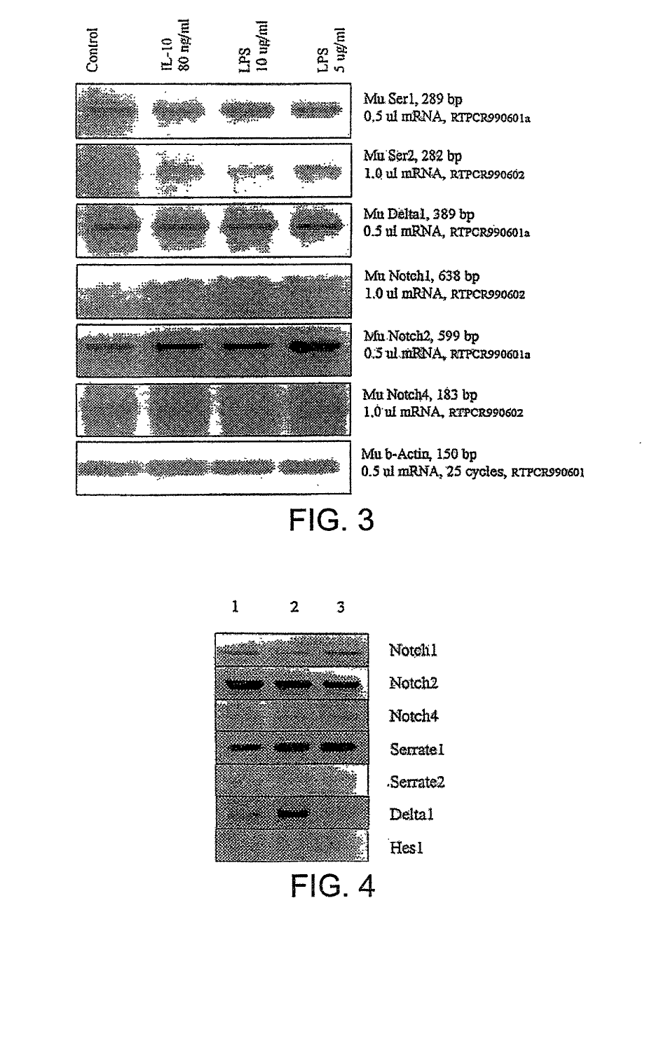

Results The results are illustrated in FIG. 3 and reveal the following changes in transcript levels for naive splenic B cells

| | | | | | |
|---|---|---|---|---|---|
| Delta1 | IL-10 | increased | Notch2 | IL-10 | increased |
| | LPS | increased | | LPS | increased |
| Serrate1 | IL-10 | no change | Notch4 | IL-10 | increased |
| | LPS | increased | | LPS | increased |

### Example 11

Modulation of the Expression of Notch Receptors and Ligands on Murine Bone Marrow Derived Dendritic Cells (DCs) in response to IL-10

CD11c+ DCs were MACS separated from the bone marrow of naive BALB/C mice and incubated at 37° C. for 24 hours in medium alone or together with IL-10 (50 ng/ml). Cells were collected and mRNA was isolated using Oligotex following the manufacturer's instructions. RT/PCR was performed using an Access RT-PCR kit (Promega). One μg of total RNA was used in each reaction together with specific oligonucleotide primers (50 pmol) for the following genes as indicated under conditions according to the manufacturer's instructions. PCR was performed using a Hybaid machine, dynazyrne II polymerase, 1.5 mM Mg, 28-35 cycles at an annealing temperature between 56-63° C.

Results The results are illustrated in FIG. 4 and reveal the following changes in transcript levels for bone marrow derived DCs

Mature versus immature DCs

Delta1 increased

Notch2 decreased

Serrate1 increased

IL-10 stimulation IL-10 of mature DCs

Delta1 decreased

Serrate1 increased

In FIG. 4 the results show bone marrow-derived DC cultivated in the presence of IL-10 immature DCs (Lane 1), mature DCs (Lane 2) and DCs cultivated with 50 ng/ml IL-10 (Lane 3). Hes1 transcription is measured as an index of Notch signalling.

### REFERENCES

Morgenstern and Land (1990) Nucleic Acids Res. 18 :3587-3596.

Cosset et al. (1995) J. Virol. 69: 7430-7436.

Coffin RS, et al. (1998) Gene Therapy 5: 718-722.

Inaba K, et al. (1992) J. Exp. Med. 175: 1157-1167.

Caux C, et al. (1992) Nature 360: 258-261.

Sallusto F and Lanzavecchia A (1994) J. Exp. Med. 179: 1109-1118.

Zhao et al. (1995) J. Immunol 155:3904-3911.

Wilson and Hemmati-Brivanlou (1997) Neuron 18: 699-710.

Hemmati-Brivanlou and Melton (1997) Cell 88:13-17.

Valenzuela et al. (1995) J. Neurosci 15: 6077-6084.

Sasai et al. (1994) Cell 79: 779-790.

Shimizu et al (1999) J. Biol. Chem. 274(46): 32961-32969.

Iemura et al. (1998) PNAS 95: 9337-9342.

Altman et al. (1996) Science 274: 94-96.

Dkuz et al (1997) Cell 90: 271-280.

1. A method for producing a lymphocyte or antigen presenting cell (APC) having tolerance to an allergen or antigen which method comprises incubating a lymphocyte or APC obtained from a human or animal patient with (i) a composition capable of upregulating expression of an endogenous Notch or Notch ligand in the lymphocyte and/or APC and (ii) the allergen or antigen.

2. A method according to claim 1 wherein the method comprises incubating a lymphocyte or APC obtained from a human or animal patient with an APC in presence of (i) a composition capable of upregulating expression of an endogenous Notch or Notch ligand in the lymphocyte and/or APC and (ii) the allergen or antigen.

3. A method according to claim 1 for producing an APC capable of inducing in a T cell tolerance to an allergen or antigen which method comprises contacting an APC with (i) a composition capable of upregulating expression of an endogenous Notch or Notch ligand in the APC and (ii) the allergen or antigen.

4. A method according to claim 1 or claim 2 for producing ex vivo a T cell having tolerance to an allergen or antigen which method comprises incubating a T cell obtained from a human or animal patient with an antigen presenting cell (APC) in the presence of (i) a composition capable of upregulating expression of an endogenous Notch or Notch ligand in the APC and/or T cell and (ii) the allergen or antigen.

5. A method according to any one of claims 1 to 4 wherein the composition comprises a substance capable of upregulating expression of Notch or a Notch ligand selected from polypeptides and fragments thereof, linear peptides, cyclic peptides, synthetic and natural compounds including low molecular weight organic or inorganic compounds.

6. A method according to any one of claims 1 to 5 wherein the composition comprises a polypeptide selected from Noggin, Chordin, Follistatin, Xnr3, FGF and derivatives, fragments, variants and homologues thereof, and immunosuppressive cytokines, or a combination thereof.

7. A method according to claim 6 wherein the immunosuppressive cytokine is selected from IL-4, IL-10, IL-13, TGF-β and FLT3 ligand.

8. A method according to any one of the preceding claims wherein the Notch ligand is selected from Serrate, Delta and homologues thereof.

9. A method according to any one of the preceding claims wherein the APC is a dendritic cell.

10. A method for producing a lymphocyte or APC having tolerance to an allergen or antigen which method comprises incubating a lymphocyte or APC obtained from a human or animal patient with a lymphocyte or APC produced by the method of any one of the preceding claims.

11. A method according to claim 9 for producing ex vivo a T cell having tolerance to an allergen or antigen which method comprises incubating a T cell obtained from a human or animal patient with a T cell produced by the method of any one of the preceding claims.

12. Use of a lymphocyte or APC produced by the method of any one of the preceding claims in suppressing an immune response in a mammal to the allergen or antigen.

13. Use of a composition capable of upregulating expression of an endogenous Notch or Notch ligand in an APC or lymphocyte in a method of producing regulatory lymphocytes capable of suppressing the activity of other lymphocytes.

14. Use according to claim 13 wherein the composition is as defined in any one of claims 5 to 7.

15. Use according to claim 13 or 14 wherein the Notch ligand is selected from Serrate, Delta and homologues thereof.

16. Use according to any one of claims 13 to 15 wherein the APC is a dendritic cell.

17. A method of treating a patient suffering from a disease characterised by inappropriate lymphocyte activity which method comprises administering to the patient a lymphocyte produced by the method of any one of claims 1 to 9.

18. A method for producing a lymphocyte having tolerance to an allergen or antigen which method comprises incubating an APC produced by the method of claim 3 with the lymphocyte.
